SEQUENCE LISTING (4 sequences)

SEQ ID NO: 1 - DNA, 1689 bp, Rhodococcus aetherivorans I24
ttgctcgaccacgtgcacaagaagttgaagtcgaccctggacccgatcggctggggtcccgcggtgaagtcggtggccggacgcgccgtccgcaacccccaggccgtcaccgccgccacgacggaatacgcgggccggctggtgaagatccccgcggcggccacccgcgtgttcaacgccgacgatcccaagccgccgatgccgctcgacccgcgggaccgccgtttctccgacaccgcctggcgggagaaccccgcgtacttctcgctcctgcagagttatctcgcgacgcgggcctacgtcgaggaactcaccgacgccggcgccggcgatccgctgcaggacggcaaggcccgccagttcgcgaacctgatgctcgacgtgctggccccgtcgaacttcctgtggaatccgggcgtgctcacccgtgcattcgagacgggcggggcaagcctgctgcgcggcgcccgatatgccgtgcacgacgtgctcaaccgcggcggcctgccgctgaaggtggactcggacgcgttcaccgtcggcgagaacctcgcggccaccccgggcaaggtggtctatcgcaacgacctgatcgagctgatccagtacacgccgcagaccgagcaggtgcatgcggtgccgatcctcgccgcgccgccgtggatcaacaagtactacatcctcgatctcgcacccggtcgcagcctcgccgagtgggcggtccagcacggccgcaccgtgttcatgctctcgtaccggaacccggacgagtcgatgcggcacatcaccatggacgactactacgtcaacggcattgccgccgcgctggacgtggtcgaggagatcaccgggtcgccgaagatcgaggtgctgtccatctgcctcggcggcgcgatggccgcgatggccgccgcgcgcgcattcgccgtcggcgacaagcgcgtgaccgccttcaccatgctcaacaccctgctcgactacagccaggtcggggaactcgggttgctgaccgatccgtccacgctggacctcgtcgagttccggatgcggcagcagggcttcctgtccggcaaggagatggccggcagcttcgacatgatccgcgcgaaggacctcgtcttcaactactgggtctcgcggtggatgaagggcgagaagcctgcggccttcgacatcctcgcgtggaacgaggacagcacgagcatgcccgcggagatgcactcgcactacctccggtcgctgtacggccgcaacgagctggccgagggcctctacgtgctcgacggacagcccctgaacctgcacgacatcacgtgcgacacctacgtcgtcggcgcgatcaacgaccacatcgtgccctggacatcgtcgtaccaggcggtgaacctgctgggcggcgacgtgcgctacgtgctcaccaacggcgggcacgtcgccggcgcggtgaacccgcccggcaagaaggtgtggttcaaggccgtcggggcgccggacgccgagaccggctcgccgctgcccgcggatccgcaggtctgggacgacgcggccacccgctacgagcactcgtggtgggaggactggacggcctggtcgaacaagcgcgccggggagctggtgccgccgccggcaatgggcagcgccgcccacccgccgctcgaggacgctccgggcacgtacgtcttcagctga

SEQ ID NO: 2 - protein, 562 aa, Rhodococcus aetherivorans I24
MLDHVHKKLKSTLDPIGWGPAVKSVAGRAVRNPQAVTAATTEYAGRLVKIPAAATRVFNADDPKPPMPLDPRDRRFSDTAWRENPAYFSLLQSYLATRAYVEELTDAGAGDPLQDGKARQFANLMLDVLAPSNFLWNPGVLTRAFETGGASLLRGARYAVHDVLNRGGLPLKVDSDAFTVGENLAATPGKVVYRNDLIELIQYTPQTEQVHAVPILAAPPWINKYYILDLAPGRSLAEWAVQHGRTVFMLSYRNPDESMRHITMDDYYVNGIAAALDVVEEITGSPKIEVLSICLGGAMAAMAAARAFAVGDKRVTAFTMLNTLLDYSQVGELGLLTDPSTLDLVEFRMRQQGFLSGKEMAGSFDMIRAKDLVFNYWVSRWMKGEKPAAFDILAWNEDSTSMPAEMHSHYLRSLYGRNELAEGLYVLDGQPLNLHDITCDTYVVGAINDHIVPWTSSYQAVNLLGGDVRYVLTNGGHVAGAVNPPGKKVWFKAVGAPDAETGSPLPADPQVWDDAATRYEHSWWEDWTAWSNKRAGELVPPPAMGSAAHPPLEDAPGTYVFS

SEQ ID NO: 3 - DNA, 1686 bp, Rhodococcus aetherivorans I24
atgatggcccaggcacgaaccgtgatcggtgagagcgtcgaggagtcgatcgggggtggcgaggacgtcgcgccaccgaggctcgggccggccgtcggcgccctggccgacgtgttcggtcacggccgggcggtggcccggcacggcgtgtcgttcggcagggaactggcgaagatcgccgtcggccggtcgacggtggctccggcgaagggagaccgccggttcgccgactcggcgtggagtgcgaaccccgcctaccgccggctcgggcagacctacctggcggcaaccgaggccgtcgacggagtcgtcgacgaggtcggtcgcgcgatcggcccgcgacgcacggccgaggccaggttcgccgccgacatcctcaccgcggccctggccccgacgaactacctgtggaccaaccccgcggcgctgaaggaggcgttcgacaccgccggactcagcctcgcacgcggcaccaagcacttcgtctccgatctgatcgagaaccggggcatgccgtcgatggtccagcgcggcgccttcaccgtcgggaaggaccttgcggtgaccccgggtgcggtgatctcccgcgacgaggtcgccgaggtgctgcagtacaccccgaccacggagacggtccgccgccggccggtgctcgtggtgcccccgccgatcggccggtactacttcctggacctgcggccgggacgcagcttcgtcgagtacagcgtgggccggggcctgcagaccttcctgctgtcgtggcgcaatcccaccgccgagcagggcgactgggacttcgacacgtacgcgggccgggtgatccgggcgatcgacgaggtgcgggagatcaccggcagcgacgacgtgaacctgatcggtttctgcgccggcgggatcatcgccaccacggtgctcaatcaccttgccgcgcagggcgacacccgagtgcacagcatggcctatgcggtgacgatgctggacttcggcgatccggcactgctcggcgcgttcgcccggcccggcctgatccggttcgccaagggccggtcccgccgcaagggcatcatcagcgcccgcgacatggggtccgcgttcacctggatgcgcccgaacgacctggtgttcaactacgtcgtcaacaactacctcatgggtcgcaccccaccggccttcgacatcctcgcctggaacgacgacggcaccaacctgcccggcgccctgcacggtcagttcctcgacatcttccgtgacaacgtgctcgtcgagcccggccggctcgccgtgctgggcacgcccgtcgacctgaagtcgatcaccgtgcccacgttcgtctcgggcgccatcgccgaccatctgaccgcatggcgcaactgctaccgcaccacccaattgctcggtggagaaacagaattcgcgctcagcttctccgggcacatcgccagcctggtcaacccgccgggcaatccgaaggcacactactggaccgggggcacacccggcccggacccggatgcctggctcgagaacgccgagcggcagcagggcagctggtggcaggcctggtccgactgggtgctcgcccgcggcggggaggaaaccgccgcgccggacgcacccggcagtgcgcagcatcccgcgctcgacgccgctcccggccggtacgtgcgcgacctgcccgccggctga

SEQ ID NO: 4 - protein, 561 aa, Rhodococcus aetherivorans I24
MMAQARTVIGESVEESIGGGEDVAPPRLGPAVGALADVFGHGRAVARHGVSFGRELAKIAVGRSTVAPAKGDRRFADSAWSANPAYRRLGQTYLAATEAVDGVVDEVGRAIGPRRTAEARFAADILTAALAPTNYLWTNPAALKEAFDTAGLSLARGTKHFVSDLIENRGMPSMVQRGAFTVGKDLAVTPGAVISRDEVAEVLQYTPTTETVRRRPVLVVPPPIGRYYFLDLRPGRSFVEYSVGRGLQTFLLSWRNPTAEQGDWDFDTYAGRVIRAIDEVREITGSDDVNLIGFCAGGIIATTVLNHLAAQGDTRVHSMAYAVTMLDFGDPALLGAFARPGLIRFAKGRSRRKGIISARDMGSAFTWMRPNDLVFNYVVNNYLMGRTPPAFDILAWNDDGTNLPGALHGQFLDIFRDNVLVEPGRLAVLGTPVDLKSITVPTFVSGAIADHLTAWRNCYRTTQLLGGETEFALSFSGHIASLVNPPGNPKAHYWTGGTPGPDPDAWLENAERQQGSWWQAWSDWVLARGGEETAAPDAPGSAQHPALDAAPGRYVRDLPAG

(19) United States
(12) Patent Application Publication
(10) Pub. No.: US 2013/0017583 A1
(43) Pub. Date: Jan. 17, 2013

(54) METHODS FOR PRODUCING POLYHYDROXYALKANOATE COPOLYMER WITH HIGH MEDIUM CHAIN LENGTH MONOMER CONTENT

(71) Applicants: Charles Forrester Budde, Cambridge, MA (US); Chokyun Rha, Boston, MA (US)

(72) Inventors: Charles Forrester Budde, Cambridge, MA (US); Chokyun Rha, Boston, MA (US)

(73) Assignee: Massachusetts Institute of Technology, Cambridge, MA (US)

(21) Appl. No.: 13/509,101

(22) PCT Filed: Nov. 10, 2010
(86) PCT No.: PCT/US10/56196

§ 371 Date: Oct. 1, 2012

Related U.S. Application Data
(60) Provisional application No. 61/260,164, filed on Nov. 11, 2009.

(51) Int. Cl. C12P 7/62; C12N 1/15; C12N 15/60; C12N 5/10; C12N 15/63; C12N 9/88; C12N 1/21; C12N 1/19

(52) U.S. Cl. 435/135; 435/252.3; 435/254.11; 435/254.2; 435/419; 435/348; 435/325; 435/252.34; 435/252.2; 435/471; 435/468; 435/455; 435/320.1; 435/232; 536/23.2

(57) ABSTRACT

The invention relates to the production of polyhydroxyalkanoate copolymer with high 3-hydroxyhexanoate monomer content through recombinant gene expression.

### RELATED APPLICATION

This application claims the benefit under 35 U.S.C. §120 of U.S. Provisional Application Ser. No. 61/260,164, entitled “Methods for Producing Polyhydroxyalkanoate Copolymer with High Medium Chain Length Monomer Content,” filed on Nov. 11, 2009, the entire disclosure of which is incorporated by reference herein in its entirety.

### FIELD OF THE INVENTION

The invention relates to the production of polyhydroxyalkanoate copolymer with high medium chain length monomer content through recombinant gene expression.

### BACKGROUND OF THE INVENTION

Polyhydroxybutyrate, a commercially useful complex biopolymer, is an intracellular material produced by a large number of bacteria. Polyhydroxybutyrate (PHB) is it a useful biomaterial based on both the chemical and physical properties of the polymer. PHB has a variety of potential applications, including utility as a biodegradable/thermoplastic material, as a source of chiral centers for the organic synthesis of certain antibiotics, and as a matrix for drug delivery and bone replacement. In vivo, the polymer is degraded internally to hydroxybutyrate, a normal constituent of human blood. Various aspects of production of polyhydroxyalkanoates (PHA, including PHB) and copolymers are described, for example, in U.S. Pat. No. 5,534,432, U.S. Pat. No. 5,663,063, U.S. Pat. No. 5,798,235, and U.S. Pat. No. 7,202,064.

As an example, the bacterium Ralstonia eutropha is well known for accumulating high levels of polyhydroxyalkanoate (PHA) bioplastic. The wild type organism typically produces the homopolymer polyhydroxybutyrate (PHB). This polymer is produced from acetyl-CoA by the action of β-ketothiolase (PhaA), an acetoacetyl-CoA reductase (PhaB), and a polyhydroxyalkanoate synthase (PhaC). PHB is not a useful bioplastic because it is brittle and has a melting temperature near its decomposition temperature.

It has previously been demonstrated that introducing 3-hydroxyhexanoate monomers into the polymer chain yields a material that is tougher and has a lower melting temperature than PHB, thus increasing the plastic's performance and making it suitable for more applications than PHB.

U.S. Pat. No. 7,235,621 describes production of a copolymer of 3-hydroxybutyrate with 3-hydroxyhexanoate under very specific conditions. U.S. Pat. No. 7,235,621 describes that specific plant oils with high lauric acid content are required as the carbon source for the microorganisms producing the copolymer. These required oils have shorter fatty acids than are found in more common oils such as palm, soybean, and rapeseed. Even with such restrictive carbon source requirements, the highest amount of 3-hydroxyhexanoate in copolymers disclosed in U.S. Pat. No. 7,235,621 is 13.8 mol %.

U.S. Patent Publication 2009/0130731 describes production of a copolymer of 3-hydroxybutyrate with 3-hydroxyhexanoate in bacteria that recombinantly express a PHA synthase gene (phaC) and a 3-ketoacyl-ACP reductase gene (fabG). However, the highest amount of 3-hydroxyhexanoate in copolymers disclosed in U.S. Patent Publication 2009/0130731 is 4 mol %.

### SUMMARY OF THE INVENTION

The invention relates to the production of polyhydroxyalkanoate copolymer with high medium chain length monomer content through recombinant gene expression.

In some aspects, cells or organisms are provided that produce polyhydroxyalkanoate copolymer with medium chain length monomer content of at least about 4 mol % or 5 wt % using any plant oil as a carbon source. In some embodiments, the cell or organism produces copolymers of 3-hydroxybutyrate with 3-hydroxyhexanoate (poly(HB-co-HHx)) with a HHx content at least about 4 mol % or 5 wt % using any plant oil as a carbon source. In some embodiments, the normal synthesis of 3-hydroxybutyrate in the cells is disrupted. In some embodiments, genes encoding acetoacetyl-CoA reductases are deleted. In some embodiments, the cell is a Ralstonia eutropha cell and one or more of the phaB1, phaB2 and phaB3 genes is disrupted. In some embodiments, the phaB3 gene is disrupted.

In some embodiments, the cell or organism recombinantly expresses a non-endogenous PHA synthase gene. In some embodiments, the non-endogenous PHA synthase gene is an Aeromonas caviae PHA synthase gene or a Rhodococcus aetherivorans PHA synthase gene. In some embodiments, the Rhodococcus aetherivorans PHA synthase gene is a Rhodococcus aetherivorans I24 D12 PHA synthase gene that encodes SEQ ID NO:4 or a Rhodococcus aetherivorans I24 C09 PHA synthase gene that encodes SEQ ID NO:2. In some embodiments, the Rhodococcus aetherivorans I24 D12 PHA synthase gene comprises or consists of SEQ ID NO:3 and/or a wherein the Rhodococcus aetherivorans I24 C09 PHA synthase gene comprises or consists of SEQ ID NO:1 or SEQ ID NO:1 in which the start codon is changed from TTG to ATG.

In some embodiments, the cell or organism recombinantly expresses an enoyl-CoA hydratase gene. In some embodiments, the enoyl-CoA hydratase gene is an Aeromonas caviae enoyl-CoA hydratase gene or a Pseudomonas aeruginosa enoyl-CoA hydratase gene. In some embodiments, the Pseudomonas aeruginosa enoyl-CoA hydratase gene is a Pseudomonas aeruginosa phaJ1 gene (gene PA3302) or a Pseudomonas aeruginosa phaJ2 gene (gene PA1018).

In some embodiments, the non-endogenous PHA synthase gene and/or the enoyl-CoA hydratase gene are amplified. In some embodiments, the monomer content is at least about 5 mol %, at least about 6 mol %, at least about 7 mol %, at least about 10 mol %, at least about 15 mol %, or at least about 20 mol %, or more.

In some embodiments, the monomer content is at least about 6 wt %, at least about 8 wt %, at least about 10 wt %, at least about 15 wt %, at least about 10 wt %, or at least about 25 wt %, or more.

In some embodiments, the cell or organism is a bacterial cell, a fungal cell (including a yeast cell), a plant cell, an insect cell or an animal cell. In some embodiments, the cell is a Ralstonia spp., an Aeromonas spp., Rhizobium spp., Alcaligenes spp., or a Pseudomonas spp. cell. In some embodiments, the cell is a Ralstonia eutropha, Aeromonas caviae, Rhizobium japonicum, Alcaligenes eutrophus or Pseudomonas oleovorans cell. In some preferred embodiments, the cell is a Ralstonia eutropha cell.

In some embodiments, the non-endogenous PHA synthase gene and/or the enoyl-CoA hydratase gene is expressed from a plasmid.

In some embodiments, the non-endogenous PHA synthase gene and/or the enoyl-CoA hydratase gene is integrated into the genome of the cell.

In other aspects, methods for producing polyhydroxyalkanoate copolymer with high medium chain length monomer content are provided. The methods include culturing the foregoing cells or organisms to produce copolymer with medium chain length monomer content of at least about 4 mol % or 5 wt %. In some embodiments, the method includes culturing the foregoing cells or organisms to produce poly(HB-co-HHx) with a HHx content at least about 4 mol % or 5 wt %, wherein copolymers of 3-hydroxybutyrate with 3-hydroxyhexanoate (poly(HB-co-HHx)) are produced. In some embodiments, the methods further include recovering the copolymer from the cells or organisms.

In some embodiments, the amount of copolymer produced is at least about 20% of cell dry weight, at least about 30% of cell dry weight, at least about 40% of cell dry weight, at least about 50% of cell dry weight, at least about 60% of cell dry weight, or more.

In other aspects, methods for producing a cell that produces polyhydroxyalkanoate copolymer with medium chain length monomer content of at least about 4 mol % or 5 wt % are provided. The methods include recombinantly expressing at least one Rhodococcus aetherivorans PHA synthase gene in the cell. In some embodiments, the cell produces copolymers of 3-hydroxybutyrate with 3-hydroxyhexanoate (poly(HB-co-HHx)) with a HHx content at least about 4 mol % or 5 wt %. In some embodiments, the Rhodococcus aetherivorans PHA synthase gene is a Rhodococcus aetherivorans I24 D12 PHA synthase gene that encodes SEQ ID NO:4 and/or a Rhodococcus aetherivorans I24 C09 PHA synthase gene that encodes SEQ ID NO:2. In some embodiments, the Rhodococcus aetherivorans I24 D12 PHA synthase gene comprises or consists of SEQ ID NO:3 and/or a wherein the Rhodococcus aetherivorans I24 C09 PHA synthase gene comprises or consists of SEQ ID NO:1 or SEQ ID NO:1 in which the start codon is changed from TTG to ATG.

In some embodiments, the methods further include recombinantly expressing an enoyl-CoA hydratase gene. In some embodiments, the enoyl-CoA hydratase gene is an Aeromonas caviae enoyl-CoA hydratase gene or a Pseudomonas aeruginosa enoyl-CoA hydratase gene. In some embodiments, the Pseudomonas aeruginosa enoyl-CoA hydratase gene is a Pseudomonas aeruginosa phaJ1 gene (gene PA3302) or a Pseudomonas aeruginosa phaJ2 gene (gene PA1018).

In some embodiments, the non-endogenous PHA synthase gene and/or the enoyl-CoA hydratase gene are amplified.

In some embodiments, the cell is a bacterial cell, a fungal cell (including a yeast cell), a plant cell, an insect cell or an animal cell. In some embodiments, the cell is a Ralstonia spp., an Aeromonas spp., a Rhizobium spp., Alcaligenes spp. or a Pseudomonas spp. cell. In some embodiments, the cell is a Ralstonia eutropha, Aeromonas caviae, Rhizobium japonicum, Alcaligenes eutrophus or Pseudomonas oleovorans cell. In some preferred embodiments, the cell is a Ralstonia eutropha cell.

In some embodiments, the non-endogenous PHA synthase gene and/or the enoyl-CoA hydratase gene is expressed from a plasmid.

In some embodiments, the non-endogenous PHA synthase gene and/or the enoyl-CoA hydratase gene is integrated into the genome of the cell.

In some aspects, methods for producing one or more polyhydroxyalkanoate copolymers with medium chain length monomer content of at least about 4 mol % or 5 wt % are provided. The method include producing a cell according to the method of any of the foregoing methods, and culturing a population of the cells. In some embodiments, one or more of the copolymers is a copolymer of 3-hydroxybutyrate with 3-hydroxyhexanoate (poly(HB-co-HHx)). In some embodiments, the methods further include collecting one or more copolymers from the population of cells.

In some embodiments, the monomer content is at least about 5 mol %, at least about 6 mol %, at least about 7 mol %, at least about 10 mol %, at least about 15 mol %, at least about 20 mol %, or more.

In some embodiments, the monomer content is at least about 6 wt %, at least about 8 wt %, at least about 10 wt %, at least about 15 wt %, at least about 20 wt %, at least about 25 wt %, or more.

In some embodiments, the amount of copolymer produced is at least about 20% of cell dry weight, at least about 30% of cell dry weight, at least about 40% of cell dry weight, at least about 50% of cell dry weight, at least about 60% of cell dry weight, or more.

In other aspects, isolated nucleic acid molecules are provided that encode SEQ ID NO:2 or SEQ ID NO:4. In some embodiments, the isolated nucleic acid molecules include the nucleotide sequence set forth as SEQ ID NO:1 or SEQ ID NO:3. In some embodiments, isolated nucleic acid molecule has at least 80% percent identity, least 90% percent identity, at least 95% percent identity, or at least 98% percent identity, or more, with the nucleotide sequence set forth as SEQ ID NO:1 or SEQ ID NO:3.

In some embodiments, isolated polypeptides encoded by the foregoing isolated nucleic acid molecules are provided.

In some embodiments, vectors comprising the foregoing isolated nucleic acid molecule are provided.

In some embodiments, cells that recombinantly express one or more of the foregoing isolated nucleic acid molecules are provided. In some embodiments, the nucleic acid molecule(s) is expressed from a vector. In some embodiments, the nucleic acid molecule(s) is integrated into the genome of the cell.

These and other aspects of the invention, as well as various embodiments thereof, will become more apparent in reference to the drawings and detailed description of the invention.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows the structure and properties of PHA polymers. Top, poly(HB); bottom, poly(HB-co-HHx)

FIG. 2 shows the involvement of PhaA, PhaB and PhaC in the pathway of PHA copolymer synthesis.

FIG. 3 schematically shows the interaction of fatty acid β-oxidation with the pathway of PHA copolymer synthesis, and that monomers are made as a byproduct of fatty acid catabolism.

FIG. 4 schematically shows the effect of blocking PhaA and PhaB on the pathway of PHA copolymer synthesis, and that monomers are made as a byproduct of fatty acid catabolism.

FIG. 5 shows PHB synthesis by several strains having deletions of phaB1, phaB2, and/or phaB3 in fructose defined medium. Results of wild type and strains with single, double and triple mutations are shown.

FIG. 6 shows reductase activity with NADPH in several strains having deletions of phaB1, phaB2, and/or phaB3 in fructose defined medium. Results of wild type and strains with single, double and triple mutations are shown.

FIG. 7 shows reductase activity with NADH in several strains having deletions of phaB1, phaB2, and/or phaB3 in fructose defined medium. Results of wild type and strains with single, double and triple mutations are shown.

FIG. 8 shows the effect of complementing reductase mutations on the strains having phaB deletions. phaB1, phaB2, and phaB3 genes were added back individually to the genome of strain Re2115 (ΔphaB123). Another strain had fabG added back to Re2115. Results of PHB synthesis in wild type, mutant and complemented strains are shown.

FIG. 9 shows the PhaA activity of the strains described in FIG. 8.

FIG. 10 shows the molecular weight of PHB polymer produced by the strains described in FIG. 8.

FIG. 11 shows PhaB substrate specificity.

FIG. 12 shows PhaJ substrate specificity.

FIG. 13 lists several strains from the literature showing the PHA synthase used, carbon source, PHA content, and mol % HHx.

FIG. 14 lists strain constructed and results from such strains, showing the genotype (including PHA synthase used), PHA content (as a % of cell dry weight), and wt % HHx.

FIG. 15 shows the procedure for strain construction in which a constructed PHA operon (phaC_{D12}-phaA-phaJ1_{Pa}) was amplified and cloned into a plasmid, which was transformed into strain Re2133.

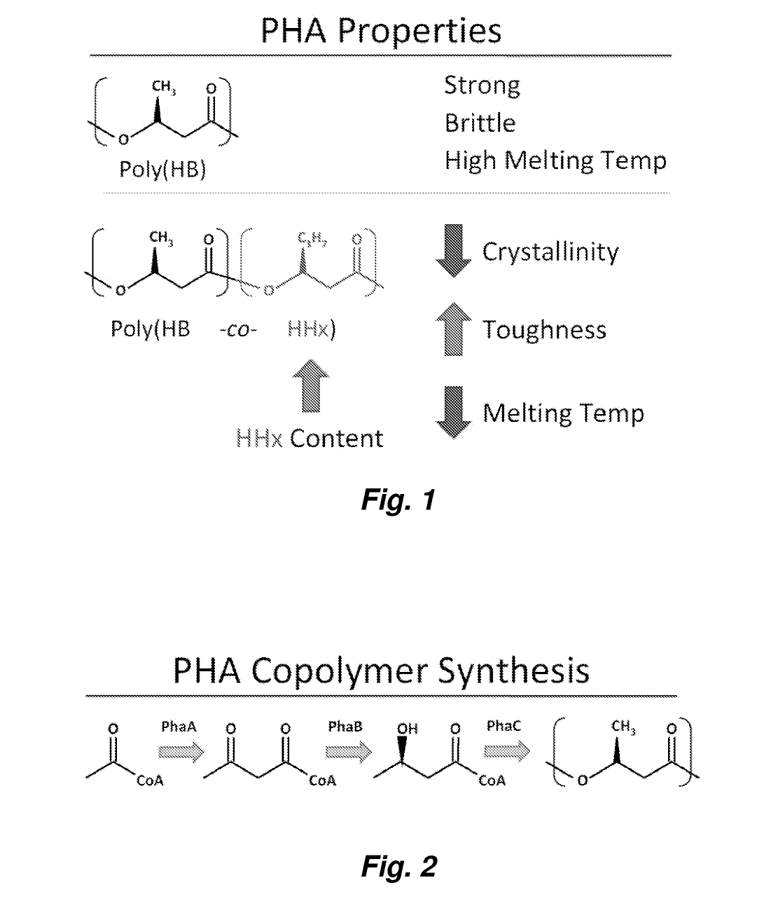

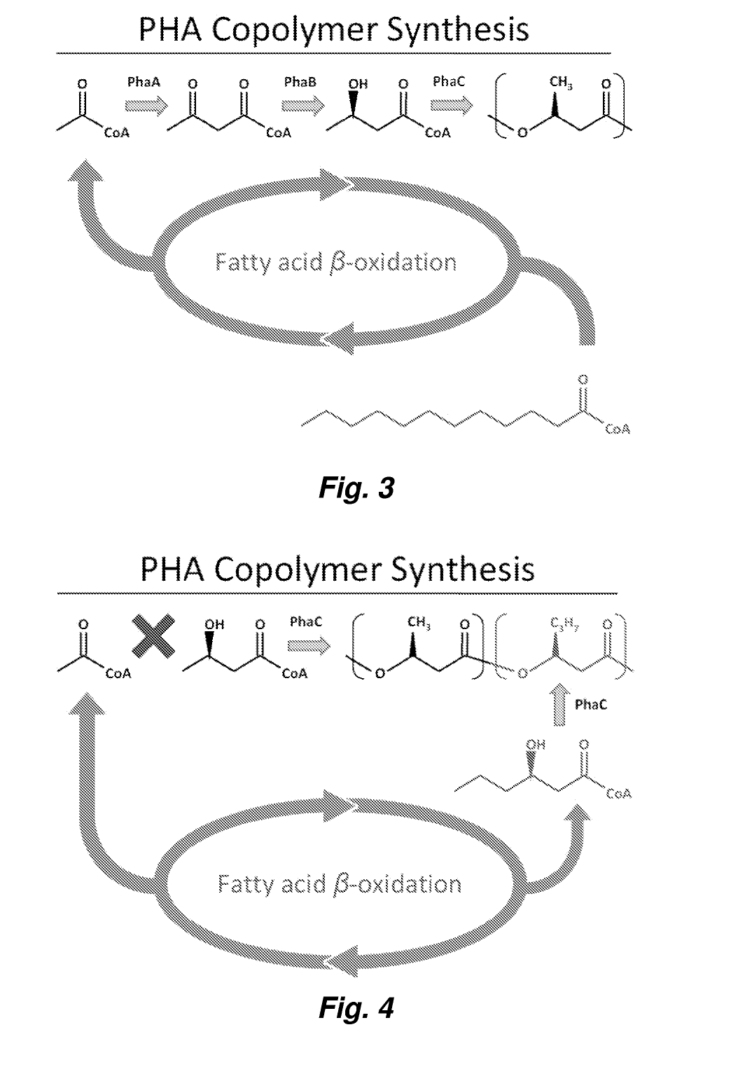

### DETAILED DESCRIPTION OF THE INVENTION

The invention relates, in part, to methods to produce bioplastics such as polyhydroxyalkanoate copolymers with high medium chain length monomer content and to increase production of bioplastics in bacterial fermentations and in other cells and organisms. Monomers that can be copolymerized to produce polyhydroxyalkanoate copolymers include 3-hydroxybutyrate and 3-hydroxyalkanoic acids with a carbon chain length greater than or equal to five (see, e.g., U.S. Pat. No. 7,341,856 and references cited therein, the disclosures of each of which is incorporated by reference for these teachings). As one example of this, copolymers of 3-hydroxybutyrate with 3-hydroxyhexanoate (poly(HB-co-HHx)), and particularly copolymers with high HHx content, are useful due to their physical properties. The invention also relates, in part, to cells, such as Ralstonia eutropha strains, that are capable of accumulating high levels of polyhydroxyalkanoate copolymers with high medium chain length monomer content, such as poly(3-hydroxybuyrate-co-3-hydroxyhexanoate), when the strain is grown using fatty acids or any plant oil as the carbon source. The 3-hydroxyhexanoate content of the copolymer made by such cells matches or exceeds any material produced from plant oil that has been described in the literature. In contrast, the methods described in U.S. Pat. No. 7,235,621 require the use of particular plant oils with lauric acid in constituent fatty acids in order to produce copolymer with acceptable HHx content. Even then, the HHx content of the copolymer produced using the method described in U.S. Pat. No. 7,235,621 is inferior to the HHx content of the copolymer produced using the methods described herein.

The following features are included in the invention, individually and in combination.

In some embodiments, normal synthesis of PHB in cells is disrupted. For example, as shown in the Examples below, PHB was disrupted in Ralstonia eutropha by deleting genes encoding acetoacetyl-CoA reductases. In certain embodiments of the invention, phaB3 is disrupted; phaB3 is a gene that has not previously been characterized in the literature and has not been used for this particular purpose. The invention is not limited to such embodiments, however, and thus includes other methods of disrupting normal PHB biosynthesis, including reducing expression of acetoacetyl-CoA reductases.

In some embodiments, the endogenous PHA synthase gene is disrupted and replaced with a new PHA synthase. The invention is not limited to such embodiments, however, and thus includes other methods of disrupting normal endogenous PHA synthase activity, including reducing expression of endogenous PHA synthase. For example, as shown in the Examples below, the wild type PHA synthase gene in Ralstonia eutropha was deleted and a new synthase gene was added to the strain. The new PHA synthase is able to incorporate a high fraction of 3-hydroxyhexanoate monomers. In some embodiments, the new synthase gene comes from the organism Rhodococcus aetherivorans I24 (also referred to herein as “Rhodococcus CO9 synthase” (SEQ ID NOs:1 and 2) or “Rhodococcus D12 synthase” (SEQ ID NOs:3 and 4)). The invention is not limited to such embodiments, however, and thus other PHA synthase genes that have similar abilities to incorporate a high fraction of medium chain length monomers, particularly 3-hydroxyhexanoate monomers, also can be used in a similar fashion.

In some embodiments, a specific enoyl-CoA hydratase gene is introduced into a cell to increase production of monomers to be incorporated into the copolymer. For example, as shown in the Examples below, a (R)-specific enoyl-CoA hydratase gene from Pseudomonas aeruginosa PAO1 called phaJ1 (gene PA3302) was introduced into the Ralstonia eutropha strain. The enzyme PhaJ1 produces 3-hydroxybutyryl-CoA and 3-hydroxyhexanoyl-CoA monomers that are then polymerized by the PHA synthase. A Pseudomonas aeruginosa PAO1 phaJ2 gene (gene PA1018) also can be used. The GenBank accession number of the Pseudomonas aeruginosa PAO1 genome is AE004091. The invention is not limited to such embodiments, however, and thus one or more other enoyl-CoA hydratase genes that have similar abilities to produce 3-hydroxybutyryl-CoA and 3-hydroxyhexanoyl-CoA monomers also can be used in a similar fashion.

In some embodiments, the copy number of the genes described herein can be modulated to change the amount of PHA accumulated by the cells. For example, as shown in the Examples below, initially the Rhodococcus D12 synthase and Pseudomonas aeruginosa phaJ1 genes were incorporated into the Ralstonia eutropha genome. The copy number of the genes was then increased by cloning the genes into a plasmid and introducing the plasmid into a Ralstonia eutropha strain in which the acetoacetyl-CoA reductase genes and the native PHA synthase gene had been deleted from the genome. The strain harboring the plasmid produced significantly more polymer than the strain in which the genes were only in the genome. The invention is not limited to such embodiments, however, and thus includes other methods of modulating (preferably increasing) copy number of the genes.

Aspects of the invention relate to methods and compositions for the production of polyhydroxyalkanoate copolymers with high medium chain length monomer content, such as copolymers of 3-hydroxybutyrate with 3-hydroxyhexanoate (poly(HB-co-HHx)) with high HHx content, through recombinant gene expression in cells. In one embodiment, described herein is the production of poly(HB-co-HHx) with HHx content of greater than 5 wt % (greater than 4 mol %) from plant oils as a carbon source in a cell that recombinantly expresses a non-endogenous PHA synthase and an enoyl-CoA hydratase gene and in which genes encoding acetoacetyl-CoA reductases are deleted. This system represents an efficient new method for producing poly(HB-co-HHx)) with high HHx content, which are molecules that have a wide variety of applications.

According to aspects of the invention, cell(s) that recombinantly express one or more enzymes and the use of such cells in producing polyhydroxyalkanoate copolymers with high medium chain length monomer content, such as poly(HB-co-HHx)) with high HHx content, are provided. It should be appreciated that the gene encoding such enzymes, including PHA synthase gene(s) can be obtained from a variety of sources.—As one of ordinary skill in the art would be aware, homologous genes for these enzymes can be obtained from other species and could be identified by homology searches, for example through a protein BLAST search, available at the NCBI internet site (www.ncbi.nlm.nih.gov). Such genes can be PCR amplified from DNA from any source of DNA which contains the particular genes. In some embodiments, the gene sequence is synthetic and/or codon optimized for the cell into which it is introduced. Any means of obtaining a gene encoding the enzymes as described herein is compatible with the instant invention.

Optimization of protein expression may also require in some embodiments that a gene encoding an enzyme be modified before being introduced into a cell such as through codon optimization for expression in a bacterial cell. Codon usages for a variety of organisms can be accessed in the Codon Usage Database (www.kazusa.or.jp/codon/).

The methods, enzymes, cells and organisms described herein provide for production of polyhydroxyalkanoate copolymer with high medium chain length monomer content. Monomer content frequently is expressed in mole percent (mol %). In some embodiments, copolymer is produced with medium chain length monomer content, such as 3-hydroxyhexanoate, of at least 4 mol %, 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, 21 mol %, 22 mol %, 23 mol %, 24 mol %, 25 mol %, 26 mol %, 27 mol %, 28 mol %, 29 mol %, 30 mol %, 31 mol %, 32 mol %, 33 mol %, 34 mol %, 35 mol %, or more. Copolymer with such medium chain length monomer content can be produced using, for example, any fatty acid or oil (e.g., plant oil) as carbon source.

Another way of expressing monomer content is in weight percent (wt %). Mole percent can be approximated from weight percent by multiplying the weight percent value by 0.8; similarly, weight percent can be approximated from mole percent by multiplying the mole percent value by 1.25. Thus, in some embodiments, copolymer is produced with medium chain length monomer content, such as 3-hydroxyhexanoate, of at least 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, or more. Copolymer with such medium chain length monomer content can be produced using, for example, any fatty acid or oil (e.g., plant oil) as carbon source for the organisms or cells.

In some embodiments, the amount of copolymer produced by the cells or organisms is at least about 20%, 22%, 24%, 26%, 28%, 30%, 35%, 40%, 45%, 50%, 55%, 60%, or more, of the dry weight of the cell or organism (cell dry weight).

As noted above, the methods described in U.S. Pat. No. 7,235,621 require the use of particular plant oils with certain amounts of lauric acid in constituent fatty acids in order to produce copolymer with acceptable HHx content. The maximum HHx content of the copolymers reported in U.S. Pat. No. 7,235,621 is 13.8 mol %. The methods, enzymes, cells and organisms described herein were used to produce copolymer of greater HHx content without regard to using a carbon source that has the lauric acid content in constituent fatty acids of the oil or fat as required by U.S. Pat. No. 7,235,621.

Useful molecular weights of the polymers include those greater than 10,000 Daltons, such as between about 10,000 and 4 million Daltons, and preferably between about 50,000 and 1.5 million Daltons.

In accordance with the invention, polyhydroxyalkanoate copolymer with high medium chain length monomer content are produced. Monomeric units are known in the art and include hydroxybutyrate, hydroxyvalerate, hydroxyhexanoate, hydroxyheptanoate, hydroxyoctanoate, hydroxynonanoate, hydroxydecanoate, hydroxyundecanoate, and hydroxydodecanoate units. In some embodiments, the copolymer produced includes hydroxybutyrate and hydroxyhexanoate monomer, such as poly(3-hydroxybutyrate-co-3-hydroxyhexanoate).

The invention encompasses any type of cell that expresses genes for making monomers that are polymerized by PHA synthase, including prokaryotic and eukaryotic cells. In some embodiments the cell is a bacterial cell. In some embodiments the bacterial cell is a Ralstonia spp., an Aeromonas spp., a Rhizobium spp., an Alcaligenes spp. or a Pseudomonas spp. cell. In some embodiments, the cell is a Ralstonia eutropha, Aeromonas caviae, Rhizobium japonicum, Alcaligenes eutrophus or Pseudomonas oleovorans cell. In one preferred embodiment, the cell is a Ralstonia eutropha cell. In other embodiments, the cell is a fungal cell such as a yeast cell, e.g., Saccharomyces spp., Schizosaccharomyces spp., Pichia spp., Phaffia spp., Hansenula spp., Kluyveromyces spp., Candida spp., Talaromyces spp., Brettanomyces spp., Schwanniomyces spp., Pachysolen spp., Debaryomyces spp., Yarrowia spp., and industrial polyploid yeast strains. Examples of yeast species and strains useful for producing copolymers are described in U.S. Pat. No. 7,083,972, the disclosure of which is incorporated by reference for these teachings. Other examples of fungi include Aspergillus spp., Pennicilium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In other embodiments, the cell is an algal cell, a mammalian cell or a plant cell.

It should be appreciated that some cells compatible with the invention may express an endogenous copy of one or more of the genes associated with the invention as well as a recombinant copy. In some embodiments, if a cell has an endogenous copy of one or more of the genes associated with the invention then the methods will not necessarily require adding a recombinant copy of the gene(s) that are endogenously expressed. In some embodiments the cell may endogenously express one or more enzymes from the pathways described herein and may recombinantly express one or more other enzymes from the pathways described herein.

It should be appreciated that some cells compatible with the invention may express an endogenous biochemical pathway as well as one or more recombinant genes of that pathway, a similar pathway, or a complementary pathway. For example, R. eutropha expresses genes for monomer production. As shown in the Examples, phaJ was additionally expressed in these cells to enhance monomer production, which are polymerized by PHA synthase.

Methods for the production of PHA polymers and copolymers by expression in plants of relevant enzymes for producing and/or polymerizing monomers, including the use of tissue-preferred promoters, are described, for example, in U.S. Pat. No. 5,534,432 and U.S. Pat. No. 7,341,856, each of which is incorporated herein by reference for these teachings.

Expression of genes in organisms and cells is well known in the art, including expression from gene(s) inserted in the genome of the organism or cell, and/or expression from gene(s) on one or more extrachromosomal nucleic acids, such as vectors, e.g., plasmids. Methods, materials and techniques for construction of modified genomes, extrachromosomal nucleic acids and organisms or cells containing such modified genomes or extrachromosomal nucleic acids are well known in the art, some of which methods, materials and techniques are described elsewhere herein.

In some embodiments it may be advantageous to use a cell that has been optimized for production of one or more polyhydroxyalkanoate copolymers with high medium chain length monomer content, such as poly(HB-co-HHx) copolymers. For example it may be optimal to mutate one or more components of biochemical pathways to eliminate competing pathways and produce more of the polyhydroxyalkanoate copolymer with high medium chain length monomer content, such as poly(HB-co-HHx) copolymers. For example, in some embodiments, 3HB-CoA synthesis is reduced, such as by mutating or deleting one or more genes contributing to 3HB-CoA synthesis. In some embodiment this can be accomplished by reducing acetoacetyl-CoA reductase activity. In R. eutropha for example, acetoacetyl-CoA reductase activity (PhaB) can be reduced by reducing phaB expression, for example by mutating or deleting (partially or completely) one or more phaB genes. As exemplified herein, this was accomplished by deleting the phaB1, phaB2, and phaB3 genes from the R. eutropha genome.

In some embodiments, screening for mutations that lead to enhanced production of one or more polyhydroxyalkanoate copolymer with high medium chain length monomer content, such as poly(HB-co-HHx) copolymers, may be conducted through a random mutagenesis screen, or through screening of known mutations. In some embodiments shotgun cloning of genomic fragments could be used to identify genomic regions that lead to an increase in production of one or more polyhydroxyalkanoate copolymers with high medium chain length monomer content, such as poly(HB-co-HHx) copolymers, through screening cells or organisms that have these fragments for increased production of one or more polyhydroxyalkanoate copolymers with high medium chain length monomer content, such as poly(HB-co-HHx) copolymers. In some cases one or more mutations may be combined in the same cell or organism.

Media for growing various cells described herein are well known in to the skilled person. For example, U.S. Pat. No. 5,534,432, which is incorporated herein by reference for these teachings, provides examples of such media that are suitable for growth of bacterial cells.

As is be understood by one of ordinary skill in the art, the optimal culture conditions for production of one or more hydroxyacids and copolymers thereof may be influenced by many factors including the type of cell, the growth media and the growth conditions. The culture temperature is a temperature at which the organism or cell can grow, and is preferably from 20° C. to 40° C. For example it may be 20, 21, 22, 23, 24, 25, 26, 27, 28, 29, 30, 31, 32, 33, 34, 35, 36, 37, 38, 39 or 40 degrees Celsius, or any value in between. The culture time period is not particularly limited, but may be approximately from 1 to 10 days.

Other non-limiting factors that can be varied through routine experimentation in order to optimize production of polyhydroxyalkanoate copolymer with high medium chain length monomer content, such as poly(HB-co-HHx) production, include the specific carbon source used, the type of culture media, the pH of the culture media, and the amount of time that the cells are cultured before harvesting the poly(HB-co-HHx). In some embodiments optimal production is achieved after culturing the cells for several days, such as 3-4 days. However, it should be appreciated that it would be routine experimentation to vary and optimize the above-mentioned parameters and other such similar parameters.

Organisms or cells are cultured in a culture medium containing a carbon source that permits production of copolymer. In some embodiments, any oil and/or fatty acid is used as a carbon source, such as any plant oil, fatty acid or fatty acid derivative, or combinations thereof. Examples of plant oils include palm oil, soybean oil, rapeseed oil, corn oil, cottonseed oil, peanut oil, coconut oil, and safflower oil; additional oils and fatty acids are well known in the art. The composition of the carbon source for optimal production of copolymer may depend on the particular strain of organism or cell used. Other components of culture medium include in some embodiments various combinations of nitrogen source(s), inorganic salt(s), selective components (such as antibiotics), or other nutrient source(s), etc., as will be known by the skilled person.

The liquid cultures used to grow cells associated with the invention can be housed in any of the culture vessels known and used in the art. In some embodiments large scale production in an aerated reaction vessel such as a stirred tank reactor can be used to produce large quantities of the polyhydroxyalkanoate copolymer with high medium chain length monomer content, such as poly(HB-co-HHx) polymers, associated with the invention.

Also known to the skilled person are methods for production of PHA polymers and copolymers on a continuous basis; such methods are described, for example, in U.S. Pat. No. 5,534,432, which is incorporated herein by reference for these teachings.

The PHA copolymer can be isolated from the organism, cell or culture in which it is produced by methods known in the art. One example of this is described in the Examples below, in which polymer is extracted from lyophilized cells for 48 h in chloroform. See also, for example, U.S. Pat. Nos. 5,942,597, 5,918,747, 5,899,339, 5,849,854, and 5,821,299; EP 859858A1; WO 97/07229; WO 97/07230; and WO 97/15681; each of which is incorporated by reference herein for these teachings.

As one non-limiting example, the process described in United States Patent Application 2009/0130731 may be employed. After the culture, the bacterial cells are separated from the culture medium by a centrifugal separator or the like, and the bacterial cells are washed with distilled water and methanol or the like, and dried. From the dried bacterial cells, the polyester is extracted using an organic solvent such as chloroform. Bacterial cell components are removed from this organic solvent solution including the polyester by filtration or the like, and a poor solvent such as methanol or hexane is added to the filtrate to permit precipitation of the polyester. Furthermore, the supernatant is removed by filtration or centrifugal separation, and dried. Other methods will be known to the skilled person.

The copolymers produced in accordance with the invention can be used in any of the many uses known in the art. For example, U.S. Pat. No. 7,455,999 describes a large number of uses for PHA polymers and physical properties of the polymers in the section headed “Applications for the Compositions” and the references cited therein, the disclosure of each of which is incorporated by reference for these teachings.

In some embodiments, the invention encompasses isolated or substantially purified PHA synthase nucleic acids or polypeptides, constructs and vectors containing or expressing such nucleic acids or polypeptides, and cells or organisms containing such nucleic acids, polypeptides, constructs or vectors. As disclosed herein, PHA synthases from Rhodococcus aetherivorans I24 that are able to incorporate a high fraction of 3-hydroxyhexanoate monomers have been identified and isolated by molecular cloning procedures. The isolated PHA synthases also are referred to herein as “Rhodococcus CO9 synthase” (SEQ ID NOs:1 and 2) or “Rhodococcus D12 synthase” (SEQ ID NOs:3 and 4).

An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A polypeptide (also referred to as a protein) that is substantially free of cellular material includes preparations of polypeptide having less than about 30%, 20%, 10%, 5%, (by dry weight) of contaminating polypeptide. When the polypeptide or biologically active portion thereof is recombinantly produced, preferably culture medium represents less than about 30%, 20%, 10%, or 5% (by dry weight) of chemical precursors or other components that are not the polypeptide.

Fragments and variants of the disclosed nucleic acid molecules and polypeptides encoded thereby are also encompassed by the present invention. By “fragment” is intended a portion of the nucleotide sequence of the nucleic acid molecule or a portion of the amino acid sequence of the polypeptide encoded thereby. Fragments of a nucleotide sequence may encode polypeptide fragments that retain the biological activity of the native polypeptide. Alternatively, fragments of a nucleotide sequence that are useful as hybridization probes generally do not encode polypeptide fragments that retain the biological activity of the native polypeptide. Thus, fragments of a nucleotide sequence may range from at least about 20 nucleotides, about 50 nucleotides, about 100 nucleotides, and up to the full-length nucleotide sequence encoding the polypeptides disclosed herein.

A fragment of a nucleotide sequence of the invention that encodes a biologically active portion of a polypeptide can encode at least 15, 25, 30, 50, 100, 150, 200, 250, 300, 350, 400, 450, 500 or 550 contiguous amino acids, or up to the total number of amino acids present in a full-length polypeptide (for example, 562 amino acids for SEQ ID NO: 2 or 561 amino acids for SEQ ID NO: 4). A biologically active portion of a polypeptide can be prepared by isolating a portion of one of the nucleotide sequences of disclosed herein, expressing the encoded portion of the polypeptide (e.g., by recombinant expression in vitro), and assessing the activity of the encoded portion of the polypeptide. Nucleic acid molecules that are fragments of a nucleotide sequence described herein (whether encoding a biologically active fragment of the polypeptide) comprise at least 15, 20, 30, 45, 60, 75, 100, 150, 200, 250, 300, 350, 400, 450, 500, 550, 600, 650, 700, 800, 900, 1000, 1100, 1200, 1300, 1400, 1500 or 1600 nucleotides, or up to the number of nucleotides present in a full-length nucleotide sequence disclosed herein (for example, 1689 nucleotides for SEQ ID NO: 1 or 1686 nucleotides for SEQ ID NO: 3).

By “variants” is intended sequences that are substantially similar to the sequences disclosed herein. For nucleotide sequences, conservative variants include those sequences that, because of the degeneracy of the genetic code, encode the amino acid sequence of one of the polypeptides or other enzymes involved in copolymer synthesis of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode a polypeptide as disclosed here. Generally, variants of a particular nucleotide sequence of the invention will have at least about 50%, 60%, 65%, 70%, 75%, 80%, 85%, 90%, 91%, 92%, 93%, 94%, 95%, 96%, 97%, 98%, 99% or more sequence identity to that particular nucleotide sequence as determined by sequence alignment programs described elsewhere herein using default parameters.

The nucleotide sequences described herein can be used to isolate corresponding sequences from other organisms, particularly other bacteria, but also other organisms or cells as described herein. In this manner, methods such as PCR, hybridization, and the like can be used to identify such sequences based on their sequence homology to the sequence set forth herein. Sequences isolated based on their sequence identity to the entire nucleotide sequences set forth herein, or to fragments thereof, are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded polypeptide sequences share substantial identity as defined elsewhere herein. Functions of orthologs are often highly conserved among species.

In a PCR-based approach, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed, for example, in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (such as genomic or cDNA libraries) from a chosen organism or cell. The hybridization probes may be genomic DNA fragments, cDNA fragments, RNA fragments, or other oligonucleotides, and may be labeled with any detectable marker, as is well known in the art. Thus, for example, probes for hybridization can be made by labeling synthetic oligonucleotides based on the nucleotide sequences(s) disclosed herein, or degenerates thereof. Methods for preparation of probes for hybridization and for construction of cDNA and genomic libraries are generally known in the art and are disclosed, for example, in Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length. Various techniques, methods, conditions and compositions used in hybridization under stringent hybridization conditions are well known in the art and can be found in references such as Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Isolated sequences that hybridize under stringent conditions to the PHA synthase sequences disclosed herein, or to fragments thereof, are encompassed by the present invention. In some embodiments, such sequences will be at least about 50%, 60%, 70%, 75%, 80%, 85%, 90%, 91%, 92%, 93%, 94%, 95%, 96%, 97%, 98%, 99% or more homologous with the disclosed sequences. That is, the sequence identity of sequences may share at least about 50%, 60%, 65%, 70%, 75%, 80%, 85%, 90%, 91%, 92%, 93%, 94%, 95%, 96%, 97%, 98%, 99% or more sequence identity with the disclosed sequences.

Methods of alignment of sequences for comparison are well known in the art. The determination of percent sequence identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87: 2264-2268, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). BLAST and related programs also are available (as are other sequence comparison programs) via the internet at the website of the National Center for Biotechnology Information (NCBI; see, e.g., www.ncbi.nlm.nih.gov or blast.ncbi.nlm.nih.gov/Blast.cgi). Alignments using these programs can be performed using the default parameters. The BLAST programs of Altschul et al (1990) J. Mol. Biol. 215:403 are based on the algorithm of Karlin and Altschul (1990). BLAST nucleotide searches can be performed, for example, with the BLASTN program, score=100, wordlength=12, to obtain nucleotide sequences homologous to a nucleotide sequence encoding a polypeptide described herein of the invention. BLAST protein searches can be performed with the BLASTX program, score=50, wordlength=3, to obtain amino acid sequences homologous to an amino acid sequence of a polypeptide described herein. To obtain gapped alignments for comparison purposes, Gapped BLAST (in BLAST 2.0) can be utilized as described in Altschul et al. (1997) Nucleic Acids Res. 25:3389-3402. Alternatively, PSI-BLAST (in BLAST 2.0) can be used to perform an iterated search that detects distant relationships between molecules. See Altschul et al. (1997). When utilizing BLAST, Gapped BLAST, PSI-BLAST, the default parameters of the respective programs (e.g., BLASTN for nucleotide sequences, BLASTX for proteins) can be used.

In some embodiments, one or more of the genes described herein are expressed in one or more recombinant expression vectors. Such vectors can contain the genes individually or in combination, such as in an operon arrangement. As used herein, a “vector” may be any of a number of nucleic acids into which a desired sequence or sequences may be inserted by restriction and ligation for transport between different genetic environments or for expression in a host cell. Vectors are typically composed of DNA although RNA vectors are also available. Vectors include, but are not limited to: plasmids, fosmids, phagemids, virus genomes and artificial chromosomes.

A cloning vector is one which is able to replicate autonomously or integrated in the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host cell such as a host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the enzymes of the claimed invention is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell. Heterologous expression of gene sequences to facilitate production of polyhydroxyalkanoate copolymer with high medium chain length monomer content, specifically poly(HB-co-HHx) with high HHx content, is demonstrated in the Examples section. The novel method for producing polyhydroxyalkanoate copolymer with high medium chain length monomer content such as poly(HB-co-HHx) with high HHx content can also be performed in other bacterial cells, archael cells, fungi (including yeast cells), mammalian cells, plant cells, etc.

A nucleic acid molecule that encodes the enzymes described herein can be introduced into a cell or cells using methods and techniques that are standard in the art. For example, nucleic acid molecules can be introduced by standard protocols such as transformation including chemical transformation and electroporation, transduction, particle bombardment, etc. Expressing one or more nucleic acid molecules encoding the enzymes of the claimed invention also may be accomplished by integrating the nucleic acid molecule into the genome of the cell.

### EXAMPLES

### Example 1

### Strains with Reduced PhaB Activity

The monomer 3-hydroxybutyryl-CoA (3HB-CoA) is synthesized from acetyl-CoA by a β-ketothiolase (PhaA) and an acetoacetyl-CoA reductase (PhaB). Analysis of protein sequences in the Ralstonia eutropha genome predicts many potential homologues to the well studied versions of these proteins (PhaA, H16_A1438; PhaB1, H16_A1439). In order to prevent 3HB-CoA synthesis, we cleanly deleted the genes phaB1, phaB2, and phaB3 from the R. eutropha genome.

Markerless deletions were made using a method adapted from York [1]. DNA sequences upstream and downstream of the gene of interest were amplified by PCR. The sequences were combined into a single contiguous stretch of DNA via overlap PCR. Primers used during this procedure were designed such that BamHI sites were added to the ends of the DNA, and a SwaI site was inserted between the upstream and downstream regions. This construct was cloned into the backbone of pGY46 at the BamHI sites to create a plasmid that could be used to create a markerless deletion of the gene of interest in R. eutropha [2]. The plasmid was transformed into E. coli S17-1 and introduced into R. eutropha via mating. Deletions were confirmed by PCR using diagnostic primers that hybridized upstream and downstream of the gene of interest.

A series of strains in which all phaB genes were deleted individually and in combination were constructed (see Table 1 below). These strains were then grown in defined medium in which fructose was the sole carbon source. PHB accumulation was induced by nitrogen limitation.

TABLE 1
Strain List

| Strain Designation | Genotype |
|---|---|
| R. eutropha H16 | Wild type |
| Re2106 | ΔphaB2 |
| Re2107 | ΔphaB3 |
| Re2111 | ΔphaB1 |
| Re2112 | ΔphaB1 ΔphaB2 |
| Re2113 | ΔphaB1 ΔphaB3 |
| Re2114 | ΔphaB2 ΔphaB3 |
| Re2115 | ΔphaB1 ΔphaB2 ΔphaB3 |

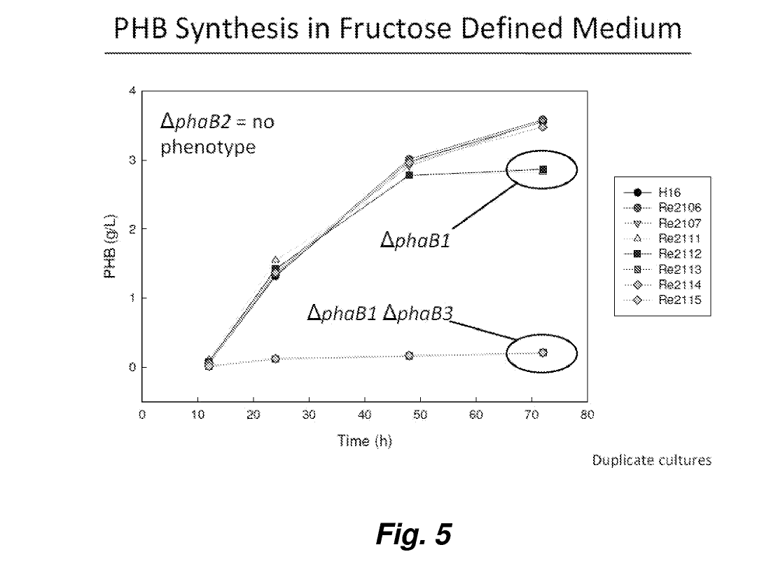

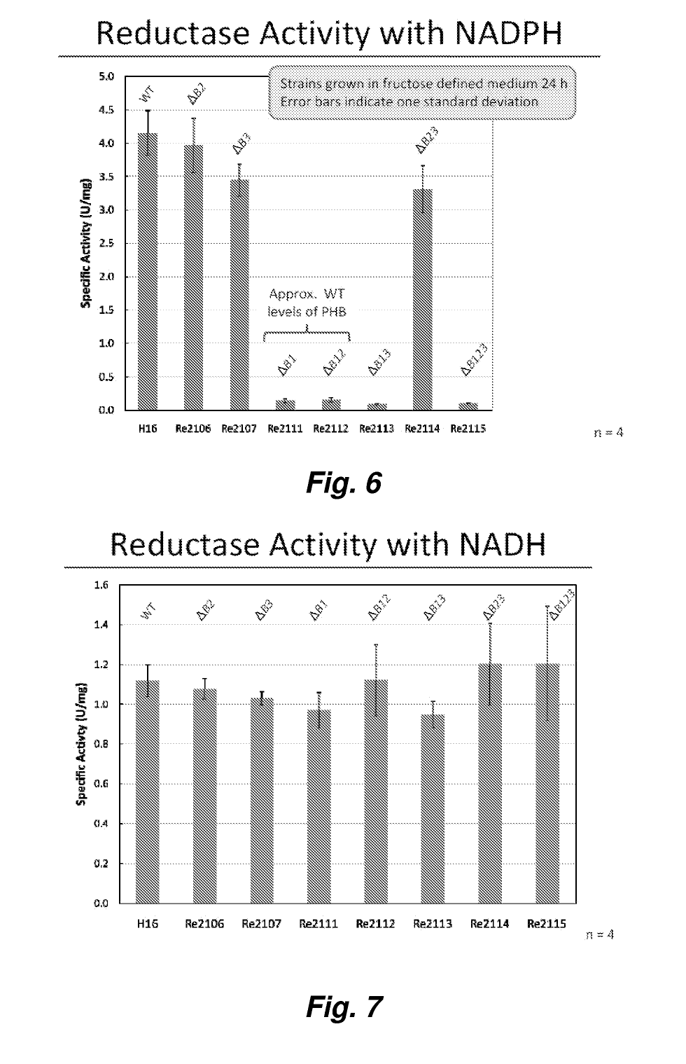

Samples from these cultures were taken at various time points and assayed for poly(3-hydroxybutyrate) (PHB) content by the crotonic acid assay [3] (FIG. 5). It was found that strains lacking both phaB1 and phaB3 (i.e. Re2113 and Re2115) produced significantly less PHB than the wild type strain. NADPH and NADH dependent acetoacetyl-CoA reductase activity was measured by the established method [4] (FIGS. 6, 7). We found that the strains that made low amounts of PHB had significantly reduced NADPH dependent reductase levels, suggesting that the decrease in PHB accumulation was indeed caused by a lack of 3HB-CoA synthesis, caused by the elimination of PhaB activity.

We examined the effect of complementing reductase mutations on the strains having phaB deletions. The phaB1, phaB2, and phaB3 genes were added back individually to the genome of strain Re2115 (ΔphaB123) at the phaB1 locus to create strains Re2139, Re2140 and Re2143. In the course of these experiments, it was discovered that the phaB3 start codon was misannotated; the correctly annotated sequence was used (denoted as phaB3 correct in Table 2). The fabG gene, encoding a reductase involved in fatty acid synthesis, also was added to strain Re2115 (ΔphaB123) at the phaB1 locus to create strain Re2142. All inserted genes have the same ribosome binding site.

TABLE 2
Strain List

| Strain | Genotype |
|---|---|
| Re2139 | Re2115 + phaB1 |
| Re2140 | Re2115 + phaB2 |
| Re2143 | Re2115 + phaB3correct |
| Re2142 | Re2115 + fabG |

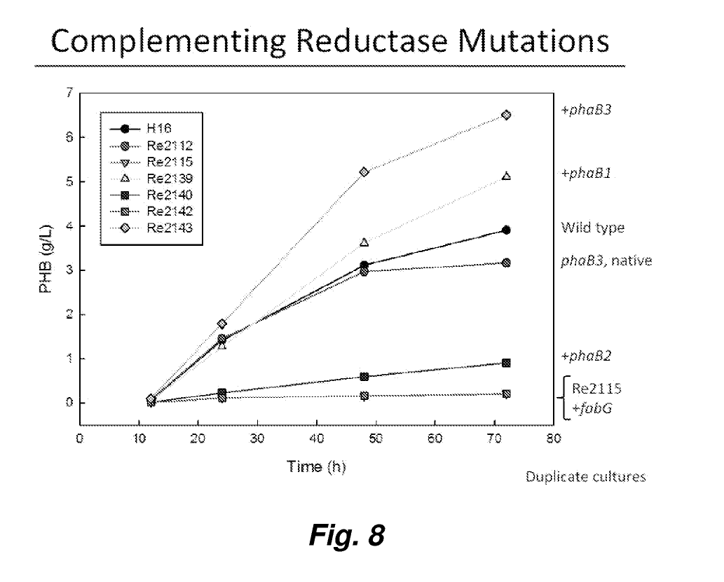

Samples from these cultures were taken at various time points and assayed for poly(3-hydroxybutyrate) (PHB) content by the crotonic acid assay [3] (FIG. 8).

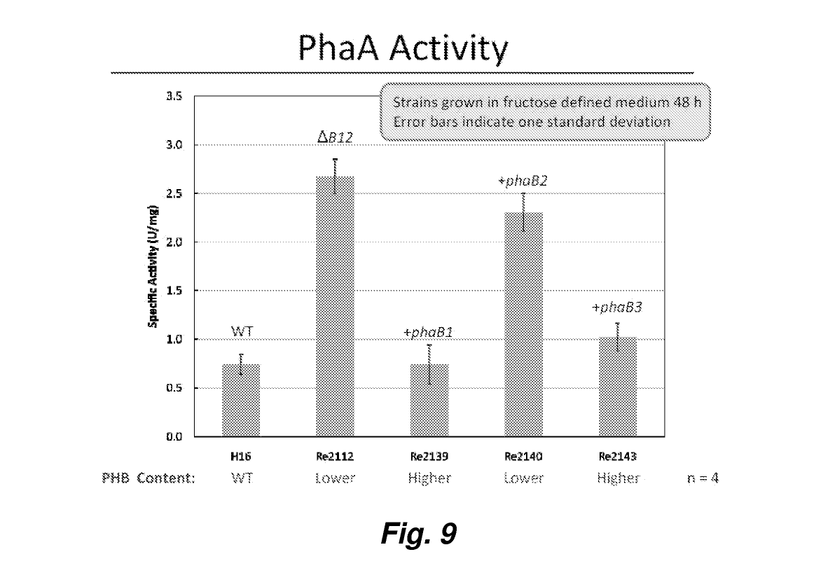

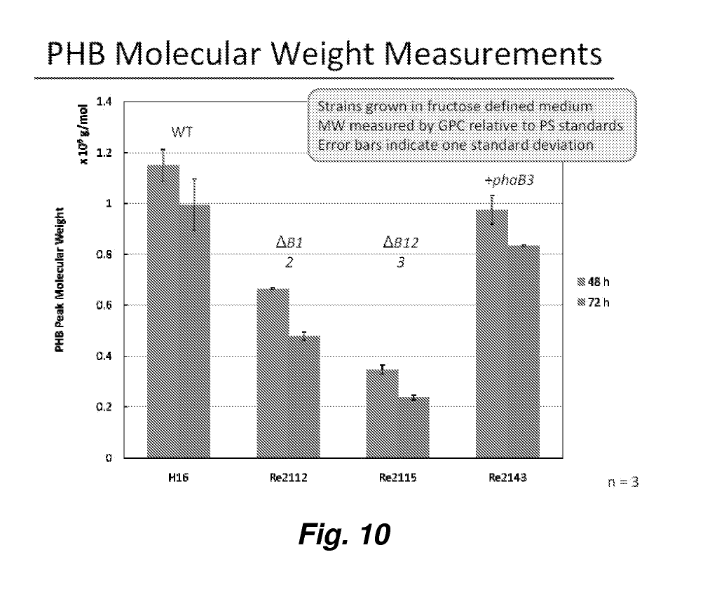

PhaA activity and molecular weight of PHB polymer produced by these strains also were measured, as shown in FIG. 9 and FIG. 10, respectively. PhaA activity was measured by the method described in [6].

Molecular weight of the copolymer was measured by gel-permeation chromatography using polystyrene standards. PHB was extracted from lyophilized cells for 48 h in chloroform. PHB solutions were prepared at concentrations of 3 mg/mL. After extraction, the solutions were filtered to remove undissolved biomass. The dissolved polymer was analyzed using an Agilent 1100 HPLC equipped with a PLgel Olexis guard column (Polymer Laboratories Part No. PL1110-1400) and two PLgel Olexis analytical columns in series (Polymer Laboratories Part No. PL1110-6400). 100 μL of each solution was injected and the polymer was detected by a refractive index detector as it eluted from the columns. Molecular weights were determined from the resulting chromatograms using Agilent GPC analysis software. The system was calibrated with a series of polystyrene standards ranging in size from 1,110 to 13,155,000 g/mol (Polymer Laboratories Part No. PL2010-0104). Isopropanol was included in all calibration standards and experimental samples as an internal standard.

### Example 2

### Strains for poly(3-hydroxybutyrate-co-3-hydroxyhexanoate) Production

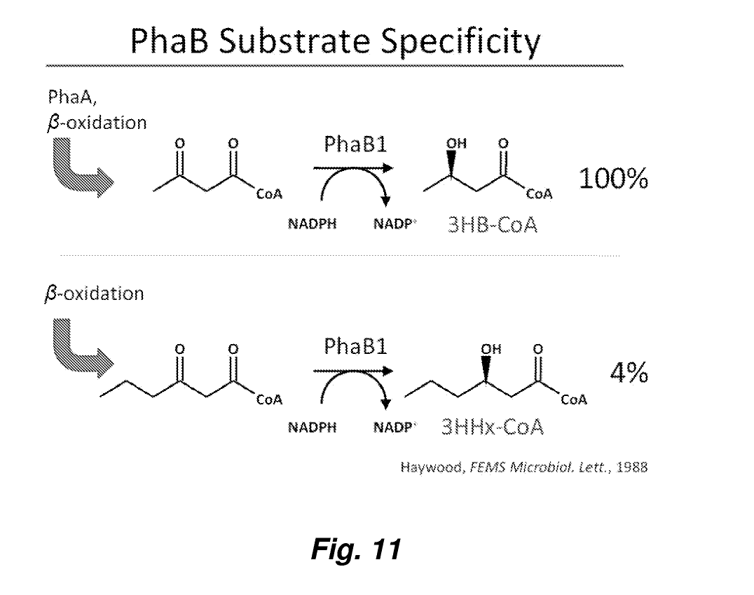

We predicted that a strain with limited ability to synthesize 3HB-CoA would be a good starting point in the design of a strain that could synthesize a copolymer of 3-hydroxybutyrate with 3-hydroxyhexanoate (poly(HB-co-HHx)) with high HHx content. This prediction was based on our belief that even if a synthase could polymerize 3HB-CoA and 3HHx-CoA, the high intracellular concentration of 3HB-CoA in wild type R. eutropha would limit HHx incorporation into the polymer. Additionally, R. eutropha synthase only polymerizes 3HB-CoA and 3HV-CoA. PhaB substrate specificity is shown in FIG. 11 (see also ref. [4]).

To test our prediction we first deleted the native PHA synthase (phaC1) from Re2115 using pGY46 [2], creating the strain Re2133. We then tested other synthases from Aeromonas caviae, and from Rhodococcus aetherivorans I24 (D12 and C09 synthases). For example, the D12 synthase from Rhodococcus aetherivorans I24 was then inserted into the Re2133 genome at the phaC1 locus using a procedure adapted from [2]. This was done by first inserting a SwaI site between the upstream and downstream regions of DNA in pGY46 via site-directed mutagenesis, and then cloning the D12 synthase gene into the SwaI site, creating strain Re2135.

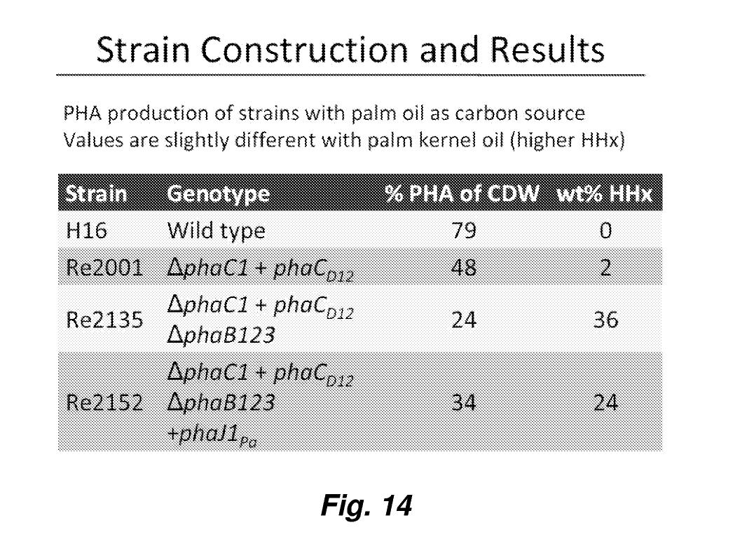

Strain Re2135 was grown in flasks using palm oil as the carbon source. We believe that 3HHx-CoA monomers are made as a byproduct of fatty acid catabolism (see FIG. 4), thus making the use of oil/fatty acids as carbon sources essential for copolymer production. PHA accumulation in these cultures was induced by nitrogen limitation. Polymer content in the cells and composition of the polymer was measured by the standard methanolysis procedure [5] (FIG. 14). This method converts PHA monomers into the related methyl esters, which are then separated and quantified by gas chromatography. We found that Re2135 accumulated a small amount of PHA, but that this PHA contained a high level of HHx monomer.

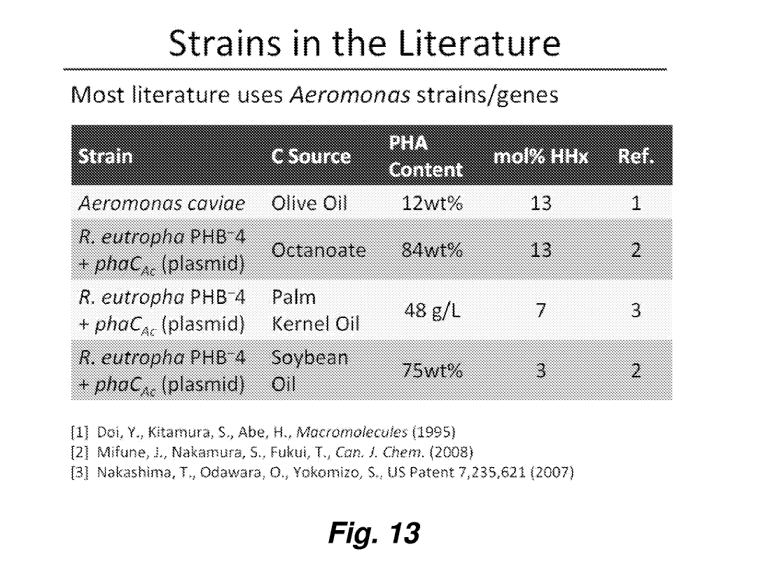

We next investigated genes for increasing monomer synthesis in this strain. Several strains from the literature are described in FIG. 13, showing the PHA synthase used, carbon source, PHA content, and mol % HHx.

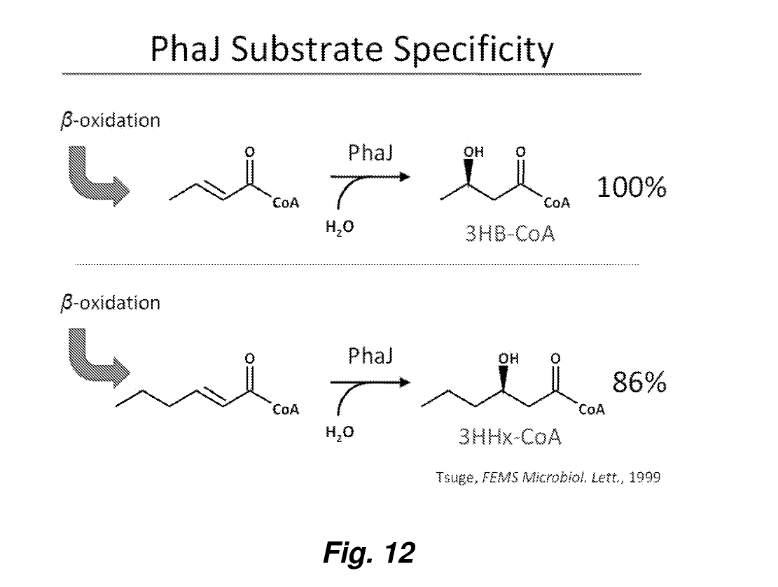

We focused on genes encoding enoyl-CoA hydratases (phaJ genes). Several phaJ's were inserted into the Re2135 genome at the phaB1 locus: enoyl-CoA hydratases from Aeromonas caviae, and from Pseudomonas aeruginosa (phaJ1 and phaJ2). PhaJ substrate specificity is shown in FIG. 12 (see also [7]).

The genes were cloned into what was originally the phaB1 deletion vector (pCB42) at the SwaI site between the upstream and downstream DNA regions. The genes were then inserted into the genome using the procedure described above, and grown in defined medium with palm oil as the sole carbon source. We found that the gene phaJ1 from Pseudomonas aeruginosa PA01 led to production of higher amounts of polymer that still contained high HHx content. The strain containing phaJ1 in the genome was named Re2152.

Strain construction and results are described in FIG. 14, showing the genotype (including PHA synthase used), PHA content (as a % of cell dry weight), and wt % HHx. HHx content measured as mol % HHx is always a lower number than HHx content measured as wt %. Mol % can be approximated from wt % by multiplying the wt % value by 0.8. For example, 25 wt % HHx corresponds to 20 mol % HHx, 30 wt % HHx corresponds to 24 mol % HHx.

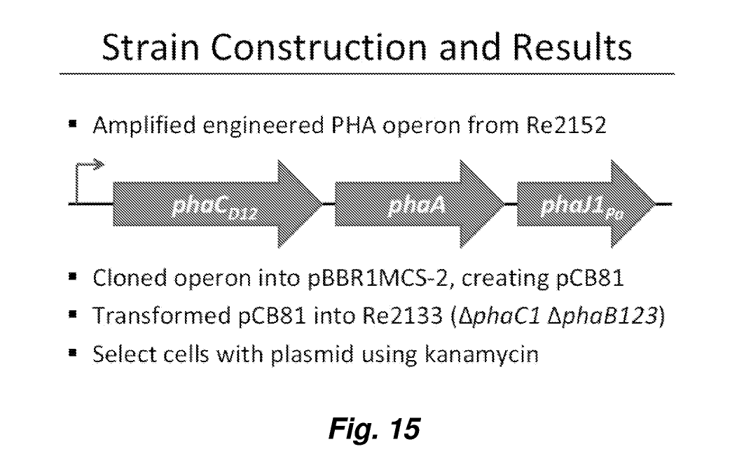

We hypothesized that one of the enzymatic steps in our newly developed PHA operon (phaC_{D12}-phaA-phanJ1_{Pa}) may have been limiting PHA production. In order to increase gene expression, we amplified this operon from Re2152 by PCR and cloned it into the plasmid pBBR1MCS-2 between the KpnI and HindIII sites. The new plasmid (pCB81) was transformed into Re2133 and the resulting strain was grown in palm oil defined medium containing 300 μg/mL of kanamycin. The procedure is described in FIG. 15. To determine if amplifying gene expression in this way negatively affected PHA production or the monomer composition of the resulting PHA, we analyzed PHA and HHX content in the polymer produced by the resulting strain. Unexpectedly, analysis of the culture showed that the cells produced a co-polymer with high PHA content and with high levels of HHx in the polymer.

We have commonly obtained copolymer with 25-30 wt % HHx, which corresponds to 20-24 mol % HHx. Copolymer with HHx values up to 33 wt % (27 mol %) have been obtained when the strains were grown on palm oil.

### Example 3

### Polymer Characterization

Properties of the PHA copolymer produced by the strain harboring the newly developed PHA operon (phaC_{D12}-phaA-phaJ1_{Pa}) were determined. Molecular weight of the PHA copolymer was found to be 120,000-150,000 g/mol relative to polystyrene standards by gel-permeation chromatography.

Thermal properties of the PHA copolymer were measured using via differential scanning calorimetry. Samples were loaded into aluminum pans and analyzed using a Perkin Elmer Pyris 1 DSC. The temperature program used was: (1) hold 1 minute at 50° C., (2) cool to −40° C. at 20° C./minute, (3) hold 3 minutes at −40° C., (4) heat to 200° C. at 20° C./minute, (5) hold 1 minute at 200° C., (6) cool to −40° C. at 20° C./minute, (7) hold 3 minutes at −40° C., (8) heat to 50° C. at 20° C./minute. Glass transition temperature was identified as the temperature at which a change in the slope of the endotherm occurred. Melting point was identified as the highest peak of the endotherm. DSC analysis revealed that a copolymer containing 27 mol % HHx, produced by the strain described above, had a glass transition temperature of −4° C.

### Example 4

### Sequence Data for Rhodococcus aetherivorans I24 PHA Synthases

Potential synthase genes were identified by BLASTing known synthase peptide sequences against the Rhodococcus aetherivorans I24 genome sequence. Based on an analysis of predicted protein sequences in the Rhodococcus aetherivorans I24 genome, two PHA synthases were cloned from the genome, which also are referred to herein as CO9 synthase and D12 synthase. Both PHA synthases were determined to be active in when expressed (as described above) in R. eutropha.

BLAST sequence analysis shows that of well studied synthases, the genes for both of the cloned PHA synthases are closest to the synthases from pseudomonads. These pseudomonad synthases are known for having very broad substrate specificity. CO9 synthase and D12 synthase will polymerize substrates up to C7-C8, which is a broader substrate specificity than the synthase from R. eutropha.

### A. CO9 Synthase

### DNA Sequence (SEQ ID NO:1)

When this gene was cloned, the start codon was changed from TTG to ATG

TTGCTCGACCACGTGCACAAGAAGTTGAAGTCGACCCTGGACCCGATCGG
CTGGGGTCCCGCGGTGAAGTCGGTGGCCGGACGCGCCGTCCGCAACCCCC
AGGCCGTCACCGCCGCCACGACGGAATACGCGGGCCGGCTGGTGAAGATC
CCCGCGGCGGCCACCCGCGTGTTCAACGCCGACGATCCCAAGCCGCCGAT
GCCGCTCGACCCGCGGGACCGCCGTTTCTCCGACACCGCCTGGCGGGAGA
ACCCCGCGTACTTCTCGCTCCTGCAGAGTTATCTCGCGACGCGGGCCTAC
GTCGAGGAACTCACCGACGCCGGCGCCGGCGATCCGCTGCAGGACGGCAA
GGCCCGCCAGTTCGCGAACCTGATGCTCGACGTGCTGGCCCCGTCGAACT
TCCTGTGGAATCCGGGCGTGCTCACCCGTGCATTCGAGACGGGCGGGGCA
AGCCTGCTGCGCGGCGCCCGATATGCCGTGCACGACGTGCTCAACCGCGG
CGGCCTGCCGCTGAAGGTGGACTCGGACGCGTTCACCGTCGGCGAGAACC
TCGCGGCCACCCCGGGCAAGGTGGTCTATCGCAACGACCTGATCGAGCTG
ATCCAGTACACGCCGCAGACCGAGCAGGTGCATGCGGTGCCGATCCTCGC
CGCGCCGCCGTGGATCAACAAGTACTACATCCTCGATCTCGCACCCGGTC
GCAGCCTCGCCGAGTGGGCGGTCCAGCACGGCCGCACCGTGTTCATGCTC
TCGTACCGGAACCCGGACGAGTCGATGCGGCACATCACCATGGACGACTA
CTACGTCAACGGCATTGCCGCCGCGCTGGACGTGGTCGAGGAGATCACCG
GGTCGCCGAAGATCGAGGTGCTGTCCATCTGCCTCGGCGGCGCGATGGCC
GCGATGGCCGCCGCGCGCGCATTCGCCGTCGGCGACAAGCGCGTGACCGC
CTTCACCATGCTCAACACCCTGCTCGACTACAGCCAGGTCGGGGAACTCG
GGTTGCTGACCGATCCGTCCACGCTGGACCTCGTCGAGTTCCGGATGCGG
CAGCAGGGCTTCCTGTCCGGCAAGGAGATGGCCGGCAGCTTCGACATGAT
CCGCGCGAAGGACCTCGTCTTCAACTACTGGGTCTCGCGGTGGATGAAGG
GCGAGAAGCCTGCGGCCTTCGACATCCTCGCGTGGAACGAGGACAGCACG
AGCATGCCCGCGGAGATGCACTCGCACTACCTCCGGTCGCTGTACGGCCG
CAACGAGCTGGCCGAGGGCCTCTACGTGCTCGACGGACAGCCCCTGAACC
TGCACGACATCACGTGCGACACCTACGTCGTCGGCGCGATCAACGACCAC
ATCGTGCCCTGGACATCGTCGTACCAGGCGGTGAACCTGCTGGGCGGCGA
CGTGCGCTACGTGCTCACCAACGGCGGGCACGTCGCCGGCGCGGTGAACC
CGCCCGGCAAGAAGGTGTGGTTCAAGGCCGTCGGGGCGCCGGACGCCGAG
ACCGGCTCGCCGCTGCCCGCGGATCCGCAGGTCTGGGACGACGCGGCCAC
CCGCTACGAGCACTCGTGGTGGGAGGACTGGACGGCCTGGTCGAACAAGC
GCGCCGGGGAGCTGGTGCCGCCGCCGGCAATGGGCAGCGCCGCCCACCCG
CCGCTCGAGGACGCTCCGGGCACGTACGTCTTCAGCTGA

### Protein Sequence (SEQ ID NO:2)

MLDHVHKKLKSTLDPIGWGPAVKSVAGRAVRNPQAVTAATTEYAGRLVKI
PAAATRVFNADDPKPPMPLDPRDRRFSDTAWRENPAYFSLLQSYLATRAY
VEELTDAGAGDPLQDGKARQFANLMLDVLAPSNFLWNPGVLTRAFETGGA
SLLRGARYAVHDVLNRGGLPLKVDSDAFTVGENLAATPGKVVYRNDLIEL
IQYTPQTEQVHAVPILAAPPWINKYYILDLAPGRSLAEWAVQHGRTVFML
SYRNPDESMRHITMDDYYVNGIAAALDVVEEITGSPKIEVLSICLGGAMA
AMAAARAFAVGDKRVTAFTMLNTLLDYSQVGELGLLTDPSTLDLVEFRMR
QQGFLSGKEMAGSFDMIRAKDLVFNYWVSRWMKGEKPAAFDILAWNEDST
SMPAEMHSHYLRSLYGRNELAEGLYVLDGQPLNLHDITCDTYVVGAINDH
IVPWTSSYQAVNLLGGDVRYVLTNGGHVAGAVNPPGKKVWFKAVGAPDAE
TGSPLPADPQVWDDAATRYEHSWWEDWTAWSNKRAGELVPPPAMGSAAHP
PLEDAPGTYVFS*

### B. D12 Synthase

### DNA Sequence (SEQ ID NO:3)

ATGATGGCCCAGGCACGAACCGTGATCGGTGAGAGCGTCGAGGAGTCGAT
CGGGGGTGGCGAGGACGTCGCGCCACCGAGGCTCGGGCCGGCCGTCGGCG
CCCTGGCCGACGTGTTCGGTCACGGCCGGGCGGTGGCCCGGCACGGCGTG
TCGTTCGGCAGGGAACTGGCGAAGATCGCCGTCGGCCGGTCGACGGTGGC
TCCGGCGAAGGGAGACCGCCGGTTCGCCGACTCGGCGTGGAGTGCGAACC
CCGCCTACCGCCGGCTCGGGCAGACCTACCTGGCGGCAACCGAGGCCGTC
GACGGAGTCGTCGACGAGGTCGGTCGCGCGATCGGCCCGCGACGCACGGC
CGAGGCCAGGTTCGCCGCCGACATCCTCACCGCGGCCCTGGCCCCGACGA
ACTACCTGTGGACCAACCCCGCGGCGCTGAAGGAGGCGTTCGACACCGCC
GGACTCAGCCTCGCACGCGGCACCAAGCACTTCGTCTCCGATCTGATCGA
GAACCGGGGCATGCCGTCGATGGTCCAGCGCGGCGCCTTCACCGTCGGGA
AGGACCTTGCGGTGACCCCGGGTGCGGTGATCTCCCGCGACGAGGTCGCC
GAGGTGCTGCAGTACACCCCGACCACGGAGACGGTCCGCCGCCGGCCGGT
GCTCGTGGTGCCCCCGCCGATCGGCCGGTACTACTTCCTGGACCTGCGGC
CGGGACGCAGCTTCGTCGAGTACAGCGTGGGCCGGGGCCTGCAGACCTTC
CTGCTGTCGTGGCGCAATCCCACCGCCGAGCAGGGCGACTGGGACTTCGA
CACGTACGCGGGCCGGGTGATCCGGGCGATCGACGAGGTGCGGGAGATCA
CCGGCAGCGACGACGTGAACCTGATCGGTTTCTGCGCCGGCGGGATCATC
GCCACCACGGTGCTCAATCACCTTGCCGCGCAGGGCGACACCCGAGTGCA
CAGCATGGCCTATGCGGTGACGATGCTGGACTTCGGCGATCCGGCACTGC
TCGGCGCGTTCGCCCGGCCCGGCCTGATCCGGTTCGCCAAGGGCCGGTCC
CGCCGCAAGGGCATCATCAGCGCCCGCGACATGGGGTCCGCGTTCACCTG
GATGCGCCCGAACGACCTGGTGTTCAACTACGTCGTCAACAACTACCTCA
TGGGTCGCACCCCACCGGCCTTCGACATCCTCGCCTGGAACGACGACGGC
ACCAACCTGCCCGGCGCCCTGCACGGTCAGTTCCTCGACATCTTCCGTGA
CAACGTGCTCGTCGAGCCCGGCCGGCTCGCCGTGCTGGGCACGCCCGTCG
ACCTGAAGTCGATCACCGTGCCCACGTTCGTCTCGGGCGCCATCGCCGAC
CATCTGACCGCATGGCGCAACTGCTACCGCACCACCCAATTGCTCGGTGG
AGAAACAGAATTCGCGCTCAGCTTCTCCGGGCACATCGCCAGCCTGGTCA
ACCCGCCGGGCAATCCGAAGGCACACTACTGGACCGGGGGCACACCCGGC
CCGGACCCGGATGCCTGGCTCGAGAACGCCGAGCGGCAGCAGGGCAGCTG
GTGGCAGGCCTGGTCCGACTGGGTGCTCGCCCGCGGCGGGGAGGAAACCG
CCGCGCCGGACGCACCCGGCAGTGCGCAGCATCCCGCGCTCGACGCCGCT
CCCGGCCGGTACGTGCGCGACCTGCCCGCCGGCTGA

### Protein Sequence (SEQ ID NO:4)

MMAQARTVIGESVEESIGGGEDVAPPRLGPAVGALADVFGHGRAVARHGV
SFGRELAKIAVGRSTVAPAKGDRRFADSAWSANPAYRRLGQTYLAATEAV
DGVVDEVGRAIGPRRTAEARFAADILTAALAPTNYLWTNPAALKEAFDTA
GLSLARGTKHFVSDLIENRGMPSMVQRGAFTVGKDLAVTPGAVISRDEVA
EVLQYTPTTETVRRRPVLVVPPPIGRYYFLDLRPGRSFVEYSVGRGLQTF
LLSWRNPTAEQGDWDFDTYAGRVIRAIDEVREITGSDDVNLIGFCAGGII
ATTVLNHLAAQGDTRVHSMAYAVTMLDFGDPALLGAFARPGLIRFAKGRS
RRKGIISARDMGSAFTWMRPNDLVFNYVVNNYLMGRTPPAFDILAWNDDG
TNLPGALHGQFLDIFRDNVLVEPGRLAVLGTPVDLKSITVPTFVSGAIAD
HLTAWRNCYRTTQLLGGETEFALSFSGHIASLVNPPGNPKAHYWTGGTPG
PDPDAWLENAERQQGSWWQAWSDWVLARGGEETAAPDAPGSAQHPALDAA
PGRYVRDLPAG*

### REFERENCES

[1] York, G., et al. (2001) New Insight into the Role of the PhaP Phasin of Ralstonia eutropha in Promoting Synthesis of Polyhydroxybutyrate. J. Bacteriol. 7, 2394-2397.
[2] York, G., et al. (2001) Accumulation of the PhaP Phasin of Ralstonia eutropha Is Dependent on Production of Polyhydroxybutyrate in Cells. J. Bacteriol. 14, 4217-4226.
[3] Karr, D., et al. (1983) Analysis of Poly-β-Hydroxybutyrate in Rhizobium japonicum Bacteroids by Ion-Exclusion High-Pressure Liquid Chromatography and UV Detection. Appl. Environ. Microbiol. 46, 1339-1344.
[4] Haywood, G., et al. (1988) The role of NADH- and NADPH-linked acetoacetyl-CoA reductases in the poly-3-hydroxybutyrate synthesizing organism Alcaligenes eutrophus. FEMS Microbiol. Lett. 52, 259-264.
[5] Brandl, H., et al. (1988) Pseudomonas oleovorans as a source of poly(beta-hydroxyalkanoates) for potential applications as biodegradable polyesters. Appl. Environ. Microb. 65, 1977-1982.
[6] Slater, S. et al. (1998) Multiple β-Ketothiolases Mediate Poly(β-Hydroxyalkanoate) Copolymer Synthesis in Ralstonia eutropha. J. Bacteriol. 180, 1979-1987.
[7] Tsuge, T., et al. (1999) Molecular cloning of two (R)-specific enoyl-CoA hydratase genes from Pseudomonas aeruginosa and their use for polyhydroxyalkanoate synthesis. FEMS Microbiol. Lett. 184, 193-198.

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety for the purposes cited herein.

We claim:

1. A cell that produces polyhydroxyalkanoate copolymer with medium chain length monomer content of at least about 4 mol % or 5 wt % using any plant oil as a carbon source.

2. The cell of claim 1, wherein the cell produces copolymers of 3-hydroxybutyrate with 3-hydroxyhexanoate (poly(HB-co-HHx)) with a HHx content at least about 4 mol % or 5 wt % using any plant oil as a carbon source.

3. The cell of claim 1, wherein the normal synthesis of 3-hydroxybutyrate in the cells is disrupted.

4. The cell of claim 3, wherein genes encoding acetoacetyl-CoA reductases are deleted.

5. The cell of claim 4, wherein the cell is a Ralstonia eutropha cell and one or more of the phaB1, phaB2 and phaB3 genes is disrupted.

6. (canceled)

7. The cell of claim 1, wherein the cell recombinantly expresses a non-endogenous PHA synthase gene.

8. The cell of claim 7, wherein the non-endogenous PHA synthase gene is an Aeromonas caviae PHA synthase gene or a Rhodococcus aetherivorans PHA synthase gene.

9-10. (canceled)

11. The cell of claim 1, wherein the cell recombinantly expresses an enoyl-CoA hydratase gene.

12. The cell of claim 11, wherein the enoyl-CoA hydratase gene is an Aeromonas caviae enoyl-CoA hydratase gene or a Pseudomonas aeruginosa enoyl-CoA hydratase gene.

13. The cell of claim 12, wherein the Pseudomonas aeruginosa enoyl-CoA hydratase gene is a Pseudomonas aeruginosa phaJ1 gene (gene PA3302) or a Pseudomonas aeruginosa phaJ2 gene (gene PA1018).

14-26. (canceled)

27. The cell of claim 1, wherein the cell is a bacterial cell, a fungal cell (including a yeast cell), a plant cell, an insect cell or an animal cell.

28. The cell of claim 27, wherein the cell is a bacterial cell or a fungal cell.

29. The cell of claim 28, wherein the cell is a Ralstonia spp., an Aeromonas spp., a Rhizobium spp., an Alcaligenes spp. or a Pseudomonas spp. cell.

30-33. (canceled)

34. A method for producing polyhydroxyalkanoate copolymer with high medium chain length monomer content, the method comprising culturing the cell of claim 1 to produce copolymer with medium chain length monomer content of at least about 4 mol % or 5 wt %.

35. The method of claim 34, wherein the copolymer is poly(HB-co-HHx) with a HHx content at least about 4 mol % or 5 wt %, and wherein copolymers of 3-hydroxybutyrate with 3-hydroxyhexanoate (poly(HB-co-HHx)) are produced.

36. The method of claim 34, further comprising recovering the copolymer from the cells.

37-41. (canceled)

42. A method for producing a cell that produces polyhydroxyalkanoate copolymer with medium chain length monomer content of at least about 4 mol % or 5 wt %, comprising recombinantly expressing at least one Rhodococcus aetherivorans PHA synthase gene in the cell.

43-56. (canceled)

57. A method for producing one or more polyhydroxyalkanoate copolymers with medium chain length monomer content of at least about 4 mol % or 5 wt %, the method comprising producing a cell according to the method of claim 42, and culturing a population of the cells.

58-76. (canceled)

77. An isolated nucleic acid molecule that encodes SEQ ID NO:2, that comprises the nucleotide sequence set forth as SEQ ID NO:1 or SEQ ID NO:1 in which the start codon is changed from TTG to ATG, or that has at least 80% percent identity with the nucleotide sequence set forth as SEQ ID NO:1.

78-82. (canceled)

83. An isolated polypeptide encoded by the nucleic acid molecule of claim 77, a vector comprising the isolated nucleic acid molecule of claim 77, or a cell that recombinantly expresses the isolated nucleic acid molecule of claim 77.

84-87. (canceled)

88. An isolated nucleic acid molecule that encodes SEQ ID NO:4, that comprises the nucleotide sequence set forth as SEQ ID NO:3, or that has at least 80% percent identity with the nucleotide sequence set forth as SEQ ID NO:3.

89-93. (canceled)

94. An isolated polypeptide encoded by the nucleic acid molecule of claim 88, a vector comprising the isolated nucleic acid molecule of claim 88, or a cell that recombinantly expresses the isolated nucleic acid molecule of claim 88.

95-98. (canceled)
